(19) United States
(12) Patent Application Publication
(10) Pub. No.: US 2005/0079193 A1
(43) Pub. Date: Apr. 14, 2005

(54) Compound for preparations for treating keratinous fibers

(76) Inventors: Claus Nieendick, Krefeld (DE); Werner Seipel, Hilden (DE)

Correspondence Address: COGNIS CORPORATION;PATENT DEPARTMENT, 300 BROOKSIDE AVENUE, AMBLER, PA, 19002, US

(21) Appl. No.: 10/925,659

(22) Filed: Aug. 25, 2004

(30) Foreign Application Priority Data
Aug. 27, 2003 (DE) 103 39 760.4

(51) Int. Cl. A61K 7/42

(52) U.S. Cl. 424/401

(57) ABSTRACT

The invention relates to wax dispersions with an average particle size of 30 μm to 100 nm containing (a) 15 to 30% by weight of a wax phase selected from dialkyl(ene)ethers, dialkyl(ene) alkoxyethers, dialkyl(ene) carbonates, fatty alcohols, ethylene glycol distearate, stearic acid monoalkyl esters, stearic acid alkoxyalkyl esters, dicarboxylic acid distearyl esters, tricarboxylic acid tristearyl esters or a mixture of these substances, (b) 1 to 10% by weight of at least one amphoteric and/or zwitterionic surfactant, (c) 7 to 15% by weight of an oil selected from di-n-octyl ether, di-n-octyl carbonate and/or a C₁₂ Guerbet alcohol and (d) water, the non-aqueous part of the dispersion making up at most 50% by weight.

### FIELD OF THE INVENTION

This invention relates to specific mixtures of certain oil components and water-containing wax dispersions which can be incorporated in cosmetic and pharmaceutical preparations, show high dermatological compatibility and may be used in particular for cosmetic hair treatment formulations.

### PRIOR ART

Surfactant-containing formulations are used in personal hygiene and body care. The use of surfactants is attended by the disadvantage that, besides their cleaning effect, they also have a defatting effect which leads, for example, to rough skin and dry hair. Because of this, so-called lipid layer enhancers are added to corresponding preparations. In the most simple case, lipid layer enhancers are fats, oils or waxes which restore the lipid content of the skin and hair.

On account of their hydrophobia, lipid layer enhancers are often difficult to incorporate in aqueous systems. One way is to melt the substances and then incorporate them in very fine distribution which, unfortunately, leads to a very elaborate processing method, even where small quantities are involved. Accordingly, another method is generally used at the formulation stage in which the lipid layer enhancers are solubilized by the use of solubilizers or hydrotropes. Unfortunately, the solubilizers or hydrotropes normally used are products containing ethylene oxide which are not readily biodegradable, adversely affect viscosity and foam generation and, in addition, contribute towards a feeling of dryness of the skin because they also dissolve the skin's own sebum in the washing process and thus counteract lipid layer enhancement.

Accordingly, a search has long been conducted to find substitutes for silicone oils which would enable cosmetics to be formulated without silicone oils without losing the specific sensory profile of those oils. Substances which are suitable as a complete or partial substitute for silicone oils in order to avoid a buildup effect on the skin and hair are known, for example, from WO 97/47281. The use of oil components selected from the group of dialkyl ethers, dialkyl cyclohexanes, Guerbet alcohols, Guerbet carbonates, ester oils, polyol polyhydroxystearates and/or hydroxycarboxylic acid esters was proposed for this purpose. WO 97/467282 describes cosmetic and/or pharmaceutical preparations containing special dialkyl carbonates and emulsifiers which are distinguished by special sensory properties, the dialkyl carbonates having proved to be equivalent substitutes for silicone oils.

WO 02/34216 discloses lipid layer enhancers in the form of concentrates containing specific fatty alcohols and certain active components selected inter alia from the group of fatty acids, oil components, fats, waxes, silicone compounds and partial glycerides. However, these known concentrates are still in need of improvement in regard to the tactile feel.

Accordingly, the problem addressed by the present invention was to provide an alternative compound for the production of cosmetic or pharmaceutical preparations which would have lipid layer enhancing properties for the skin and hair. In addition, the cosmetic or pharmaceutical preparations produced with this compound would be characterized by a good feeling on the skin and hair. The compounds would also lend themselves to cold incorporation in cosmetic and pharmaceutical formulations.

### DESCRIPTION OF THE INVENTION

The present invention relates to wax dispersions with an average particle size of 30 μm to 100 nm containing (a) 15 to 30% by weight of a wax phase selected from dialkyl(ene)ethers, dialkyl(ene) alkoxyethers, dialkyl(ene) carbonates, fatty alcohols, ethylene glycol distearate, stearic acid monoalkyl esters, dicarboxylic acid distearyl esters, tricarboxylic acid tristearyl esters, stearic acid alkoxyalkyl esters and/or a mixture of these substances, (b) 1 to 10% by weight of at least one amphoteric and/or zwitterionic surfactant, (c) 7 to 15% by weight of an oil selected from di-n-octyl ether, di-n-octyl carbonate and/or a C₁₂ Guerbet alcohol and (d) water, the non-aqueous part of the dispersion making up at most 50% by weight.

Wax Phase

The wax phase may be selected from dialkyl(ene) ethers, dialkyl(ene) alkoxyethers, dialkyl(ene) carbonates, fatty alcohols, ethylene glycol distearate, stearic acid monoalkyl esters, stearic acid alkoxyalkyl esters, dicarboxylic acid distearyl esters, tricarboxylic acid tristearyl esters or a mixture of these substances. Compounds containing a stearyl residue are preferred because they have a melting point of 50 to 75° C. Waxes with a melting point of 55 to 65° C. are particularly preferred. In a preferred embodiment, the waxes phase is selected from the group of compounds consisting of distearylether, PEG-4 distearyl ether, distearyl carbonate, stearyl stearate, PEG-3 stearic acid stearyl ester, adipic acid distearyl ether, azelaic acid distearyl ether, tristearyl citrate or mixtures of these compounds. In a particularly preferred embodiment, the wax phase is an ethylene glycol distearate which is characterized in that 70 to 85% of the fatty acid component of the ethylene glycol distearate consists of octadecanoic acid. In another particularly preferred embodiment, the wax phase is a stearic acid alkoxyalkyl ester, the alcohol component of the stearic acid alkoxyalkyl ester—of which at least 90% by weight consists of stearyl alcohol—having been etherified with ethylene oxide.

The wax dispersions according to the invention typically contain 15 to 30% by weight, preferably 10 to 25% by weight and, in a particularly preferred embodiment, 20 to 25% by weight of a wax phase.

Amphoteric and/or Zwitterionic Surfactants

Typical examples of amphoteric or zwitterionic surfactants are alkyl betaines, alkylamidobetaines, aminopropionates, aminoglycinates, imidazolinium betaines and sulfobetaines. Alkylamidobetaines and especially cocamidopropyl betaine are preferred for the purposes of the invention. Wax dispersions according to the invention contain 1 to 10% by weight, preferably 2 to 7% by weight and more particularly 3 to 5% by weight of amphoteric or zwitterionic surfactants.

Oils

According to the invention, di-n-octylether, di-n-octyl carbonate and/or C₁₂ Guerbet alcohols (preferably ethyl decanol) in quantities of 7 to 15% by weight and preferably 10 to 13% by weight are used as component (c).

Polymers

In another preferred embodiment, the wax dispersions according to the invention contain at least one polymer in a quantity of 0.01 to 5% by weight, based on the wax dispersion. The polymer is preferably selected from the group of polyacrylates, polysaccharides, polyacrylamides or a mixture of these polymers. Cationically modified polysaccharides are particularly preferred.

The non-aqueous part of the wax dispersions according to the invention makes up at most 50% by weight and preferably 35 to 45% by weight.

Cosmetic/Pharmaceutical Preparations

The wax dispersions according to the invention may be incorporated in cosmetic and/or pharmaceutical preparations, more particularly in body care and hair care preparations. They are intended to impart lipid layer enhancing properties to these preparations so that they have a good feeling on the skin and hair. In this case, the preparations are, for example, body care products formulated as creams, milks, lotions or sprayable emulsions, products for eliminating body odor or UV protection products. The composition according to the invention may also be used in surfactant-containing formulations such as, for example, foam and shower baths, hair shampoos, hair treatments and care rinses. Preparations such as these are preferably free from silicones. Accordingly, the present invention also relates to the use of the wax dispersions in body care and/or hair care preparations.

The cosmetic preparations may be formulated as emulsions or dispersions which contain water and oil phase alongside one another. Preferred cosmetic compositions are those in the form of a w/o or o/w emulsion with the usual concentrations—familiar to the expert—of oils/fats/waxes, emulsifiers, water and the other auxiliaries and additives typically found in cosmetic preparations.

Depending on their intended application, the cosmetic formulations contain a number of other auxiliaries and additives such as, for example, surface-active substances (surfactants, emulsifiers), other oil components, pearlizing waxes, consistency factors, thickeners, superfatting agents, stabilizers, polymers, silicone compounds, fats, waxes, lecithins, phospholipids, biogenic agents, UV protection factors, antioxidants, deodorizers, antiperspirants, antidandruff agents, film formers, swelling agents, insect repellents, self-tanning agents, tyrosinase inhibitors (depigmenting agents), hydrotropes, solubilizers, preservatives, perfume oils, dyes and the like which are listed in the following. The quantities in which the particular additives are used are determined by the intended application.

Surface-Active Substances

The surface-active substances present may be anionic, nonionic, cationic and/or amphoteric or zwitterionic surfactants or emulsifiers or a mixture of these surfactants/emulsifiers. The content of surface-active substances is determined by the type of formulation, but does not normally exceed 20% by weight. Surfactant-containing cosmetic preparations, for example shower gels, foam baths, shampoos, etc., preferably contain at least one anionic surfactant. Body care creams and lotions preferably contain nonionic surfactants/emulsifiers.

Typical examples of anionic surfactants are soaps, alkyl benzene-sulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, polyglycerol esters, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, amino-propionates, aminoglycinates, imidazolinium betaines and sulfobetaines. The surfactants mentioned are all known compounds. Information on their structure and production can be found in relevant synoptic works. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Body care preparations, such as creams, lotions and milks, normally contain a number of other oil components and emollients which contribute towards further optimization of their sensory properties. The oil components are present in a total quantity of typically 1 to 50% by weight, preferably 5 to 25% by weight and more particularly 5 to 15% by weight. Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, Guerbet carbonates based on fatty alcohols containing 6 to 18 and preferably 8 to 10 carbon atoms, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols.

Fats and Waxes

Fats and waxes are added as care components to the body care products and also to increase the consistency of the cosmetics. Typical examples of fats are glycerides, i.e. solid or liquid, vegetable or animal products which consist essentially of mixed glycerol esters of higher fatty acids. Fatty acid partial glycerides, i.e. technical mono- and/or diesters of glycerol with C₁₂₋₁₈ fatty acids, such as for example glycerol mono/dilaurate, palmitate or stearate, are also suitable for this purpose. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Thickeners

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl and hydroxypropyl cellulose, polyacrylates (for example Carbopols® and Pemulen types [Goodrich]; Synthalens® [Sigma]; Keltrol types [Kelco]; Sepigel types [Seppic]; Salcare types [Allied Colloids] and Cosmedia® SP and SPL [Cognis]), polyacrylamides, polymers, polyvinyl alcohol, polyvinyl pyrrolidone and bentonites such as, for example, Bentone® GelVS-5PC (Rheox). Electrolytes, such as sodium chloride and ammonium chloride, are also suitable thickeners.

Stabilizers

Metal salts of fatty acids such as, for example, magnesium, aluminium and/or zinc stearate or ricinoleate may be used as stabilizers.

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. Typical UV-A filters are, in particular, derivatives of benzoyl methane. The UV-A and UV-B filters may of course also be used in the form of mixtures, for example combinations of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene), and esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide. Silicates (talcum), barium sulfate and zinc stearate may be used as salts. The oxides and salts are used in the form of the pigments for skin-care and skin-protecting emulsions.

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin.

Biogenic Agents

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prune extract, bambara nut extract, and vitamin complexes.

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups.

Preservatives

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, and synthetic perfume compounds of the ester, ether, aldehyde, ketone, alcohol and hydrocarbon type, are also suitable.

Dyes

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). These dyes are normally used in concentrations of 0.001 to 0.1% by weight, based on the mixture as a whole.

### EXAMPLES

The following cosmetic preparations were produced using the wax dispersions according to the invention and are distinguished by an excellent skin and hair feel and by good lipid layer enhancing properties. All quantities are percentages by weight.

| Ingredients | 1 | | | | | |
|---|---|---|---|---|---|---|
| INCI | Placebo | 2 | 3 | 4 | 5 | 6 |
| Texapon N 70 | 13.0 | 13.0 | 13.0 | 13.0 | 13.0 | 13.0 |
| Sodium laureth sulfate | | | | | | |
| Dehyton PK 45 | 4.5 | 7.5 | 7.5 | 7.5 | 7.5 | 7.5 |
| Cocamidopropyl betaine | | | | | | |
| Carbopol Aqua SF-1 | 3.0 | 3.0 | 3.0 | 3.0 | 3.0 | 2.9 |
| Acrylate polymer | | | | | | |
| Polymer JR 400 | — | 0.2 | 0.2 | 0.2 | 0.2 | 0.2 |
| Polyquaternium-10 | | | | | | |
| Lamesoft KE 3999 | 5.0 | — | — | — | — | — |
| 20% by weight Ethylene glycol distearate | | | | | | |
| (and) 10% by weight Cocamidopropyl betaine | | | | | | |
| (and) 5% by weight Dioctyl ether | | | | | | |
| Lamesoft KE 4003 | — | — | — | — | — | 5.0 |
| 20% by weight Ethylene glycol distearate | | | | | | |
| (and) 10% by weight Cocamidopropyl betaine | | | | | | |
| (and) 8% by weight Dioctyl ether | | | | | | |
| 1% Carbopol Aqua SF1 | | | | | | |
| Lamesoft KE 3809 | — | 5.0 | 5.0 | — | — | — |
| 20% by weight Distearyl carbonate (and) | | | | | | |
| 10% by weight Cocamidopropyl betaine | | | | | | |
| (and) 3% by weight Sodium laureth sulfate | | | | | | |
| (and) 8% by weight Dioctyl carbonate (and) | | | | | | |
| 2% by weight Octyldodecanol | | | | | | |
| Lamesoft KE 3890 | — | — | — | 5.0 | 5.0 | — |
| 10% by weight Distearyl carbonate (and) | | | | | | |
| 10% by weight Stearyl stearate (and)10% by | | | | | | |
| weight Cocobetaine (and) 5% by weight | | | | | | |
| Cocoglucoside (and) 8% by weight | | | | | | |
| Dioctylether | | | | | | |
| Perfume | 0.3 | 0.3 | 0.3 | 0.3 | 0.3 | 0.3 |
| Preservative | 0.1 | 0.1 | 0.1 | 0.1 | 0.1 | 0.1 |
| Sodium chloride | 1.6 | 1.5 | 1.3 | 1.3 | 0.1 | 0.1 |
| Water | to 100 | | | | | |
| pH value | 6.49 | 6.5 | 6.52 | 6.52 | 6.5 | 6.45 |

Dry and wet combability were determined for formulations 1 to 8 mentioned above.

In order to determine combing work, bleached hair tresses were treated with the product to be tested (1 g product/1 g hair), contact time 5 mins., and then rinsed (tap water 38° C., 1 l/min.). The forces generated during combing of the hair were then determined in a special measuring apparatus. The combing work was determined by integration from the measured force/stroke curves. The quotient of combability for untreated hair tresses and combability for treated hair tresses gives the so-called residual combing work. Each measurement was conducted on 20 hair tresses.

18 cm long hair tresses (2 g) conditioned for 12 h in the closed apparatus before the measurement were used for determining dry combability. Combing was carried out with combs having a spacing between teeth of 2.6 mm. 15 cm long hair tresses (1 g) were used for wet combability, the measurement being carried out immediately after rinsing. The combs used had a spacing between teeth of 0.7 mm. To guarantee constant conditions, the entire apparatus was accommodated in a housing where the temperature was 30° C. and the relative air humidity 40%.

| | Wet combing | Wet combing work | Residual combing |
|---|---|---|---|
| Formulation | work before [mJ] | after [mJ] | work [%] |
| 1 Placebo | 108.51 | 100.54 | 92.66 |
| 2 | 100.32 | 61.11 | 60.09 |
| 3 | 106.55 | 63.77 | 59.85 |
| 4 | 108.25 | 62.40 | 57.64 |
| 5 | 102.60 | 60.10 | 58.57 |
| 6 | 105.33 | 66.63 | 63.26 |

The series of measurements differ with 95% significance in relation to wet combing work before.

| | Wet combing | Wet combing work | Residual combing |
|---|---|---|---|
| Formulation | work before [mJ] | after [mJ] | work [%] |
| 1 Placebo | 79.51 | 67.9 | 85.39 |
| 2 | 76.91 | 50.30 | 63.10 |
| 3 | 74.60 | 48.75 | 65.34 |
| 4 | 78.25 | 49.05 | 62.68 |
| 5 | 72.94 | 45.15 | 61.90 |
| 6 | 77.88 | 50.40 | 64.71 |

The series of measurements differ with 95% significance in relation to wet combing work before.

The wet and dry combability results show that the combinations according to the invention produce the greatest reduction in combing work.

1-8. (canceled)

9. A wax dispersion comprising:
(a) from about 15 to 30% by weight of a wax component selected from the group consisting of dialkyl(ene) ethers, dialkyl(ene) alkoxyethers, dialkyl(ene) carbonates, fatty alcohols, ethylene glycol distearate, stearic acid monoalkyl esters, dicarboxylic acid distearyl esters, tricarboxylic acid tristearyl esters, stearic acid alkoxyalkyl esters, and mixtures thereof;
(b) from about 1 to 10% by weight of a surfactant selected from the group consisting of an amphoteric surfactant, a zwitterionic surfactant, and mixtures thereof;
(c) from about 7 to 15% by weight of an oil component selected from the group consisting of di-n-octyl ether, di-n-octyl carbonate, a C₁₂ Guerbet alcohol, and mixtures thereof;
(d) optionally, a polymer; and
(e) remainder, to 100% by weight, water, and wherein the wax dispersion has an average particle size of from about 30 μm to 100 nm, and the water is present in an amount of up to 50% by weight, based on the weight of the dispersion.

10. The dispersion of claim 9 wherein (a) is present in the dispersion in an amount of from about 20 to 25% by weight, based on the weight of the dispersion.

11. The dispersion of claim 9 wherein (b) is present in the dispersion in an amount of from about 3 to 5% by weight, based on the weight of the dispersion.

12. The dispersion of claim 9 wherein (c) is present in the dispersion in an amount of from about 10 to 13% by weight, based on the weight of the dispersion.

13. The dispersion of claim 9 wherein (d) is present in the dispersion in an amount of from about 0.01 to 5% by weight, based on the weight of the dispersion.

14. The dispersion of claim 9 wherein (e) is present in the dispersion in an amount of from about 35 to 45% by weight, based on the weight of the dispersion.

15. A process for treating human skin or hair comprising contacting the skin or hair with a cosmetic composition containing a wax dispersion, the wax dispersion comprising:
(a) from about 15 to 30% by weight of a wax component selected from the group consisting of dialkyl(ene) ethers, dialkyl(ene) alkoxyethers, dialkyl(ene) carbonates, fatty alcohols, ethylene glycol distearate, stearic acid monoalkyl esters, dicarboxylic acid distearyl esters, tricarboxylic acid tristearyl esters, stearic acid alkoxyalkyl esters, and mixtures thereof;
(b) from about 1 to 10% by weight of a surfactant selected from the group consisting of an amphoteric surfactant, a zwitterionic surfactant, and mixtures thereof;
(c) from about 7 to 15% by weight of an oil component selected from the group consisting of di-n-octyl ether, di-n-octyl carbonate, a C₁₂ Guerbet alcohol, and mixtures thereof;
(d) optionally, a polymer; and
(e) remainder, to 100% by weight, water, and wherein the wax dispersion has an average particle size of from about 30 μm to 100 nm, and the water is present in an amount of up to 50% by weight, based on the weight of the dispersion.

16. The dispersion of claim 15 wherein (a) is present in the dispersion in an amount of from about 20 to 25% by weight, based on the weight of the dispersion.

17. The dispersion of claim 15 wherein (b) is present in the dispersion in an amount of from about 3 to 5% by weight, based on the weight of the dispersion.

18. The dispersion of claim 15 wherein (c) is present in the dispersion in an amount of from about 10 to 13% by weight, based on the weight of the dispersion.

19. The dispersion of claim 15 wherein (d) is present in the dispersion in an amount of from about 0.01 to 5% by weight, based on the weight of the dispersion.

20. The dispersion of claim 15 wherein (e) is present in the dispersion in an amount of from about 35 to 45% by weight, based on the weight of the dispersion.

21. A cosmetic composition comprising the wax dispersion of claim 9.
